(19) United States
(12) Patent Application Publication
(10) Pub. No.: US 2011/0021850 A1
(43) Pub. Date: Jan. 27, 2011

(54) RENEWABLE BASE OIL COMPOSITION

(76) Inventor: Nigel Stewart Battersby, Ince Chester Cheshire (GB)

Correspondence Address: SHELL OIL COMPANY, P O BOX 2463, HOUSTON, TX, 772522463, US

(21) Appl. No.: 12/745,568

(22) PCT Filed: Dec. 4, 2008
(86) PCT No.: PCT/EP08/66800

§ 371 Date: Oct. 7, 2010

(30) Foreign Application Priority Data
Dec. 7, 2007 (EP) 07122614.6

(51) Int. Cl. C07C 9/22; C07C 5/03

(52) U.S. Cl. 585/16; 585/310

(57) ABSTRACT

The present invention relates to a base oil composition comprising at least one or more hydrogenated polymethylated triterpenes of the general formula CₙH₍₂ₙ₊₂₎.

The present invention relates to a renewable base oil composition, to lubricating compositions comprising the base oil, and to a process to prepare the base oil and lubricant composition.

Suitable feedstocks for paraffinic base oils are getting scarce with the exhaustion of light paraffinic crude oils such as North Sea crudes. Hence there is a need for a source for raw materials based on alternative resources. At the same time, the use of raw materials derived from renewable sources is highly desirable since this contributes to reducing the carbon footprint of such products.

Applicants have now found novel base oil compositions derived from living algae, as well as a process to prepare such base oils from the hydrocarbons obtainable from the living alga Botryococcus braunii (further referred to as B. braunii). The hydrocarbons obtainable from B. braunii may be employed as basis for a base oil composition for use in lubricant compositions, which exhibit a high oxidation stability and good overall lubricant base oil properties.

### SUMMARY OF THE INVENTION

Accordingly, the present invention relates to a base oil composition comprising at least one or more hydrogenated polymethylated triterpenes (known as ‘botryococcenes’) of the general formula CₙH₍₂ₙ₋₁₀₎. The hydrogenated botryococcenes are known as ‘botryococcanes’.

### DETAILED DESCRIPTION OF THE FIGURES

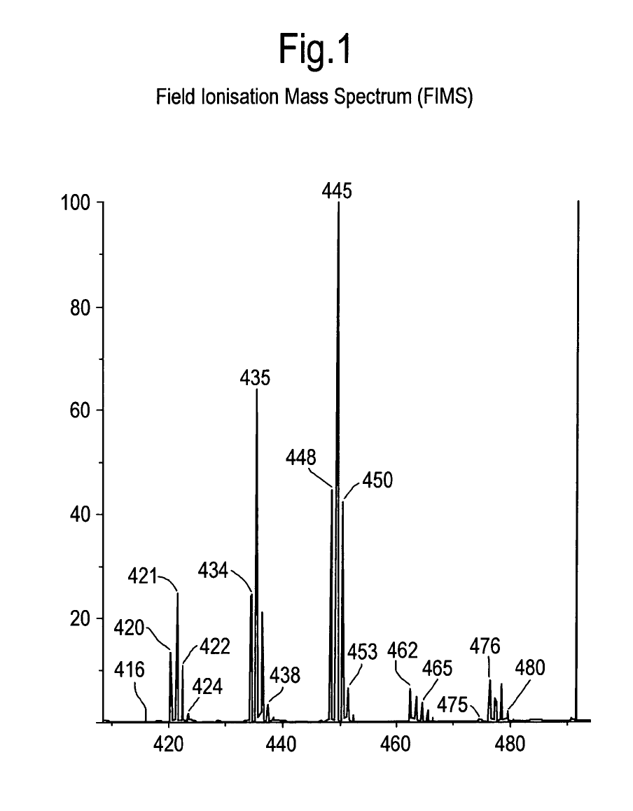

FIG. 1 shows the mass spectrum of the hydrogenated botryococcene sample obtained using field ionisation (FIMS). The spectrum shows three main groups of ions at masses: 448, 449 and 450; 434, 435 and 436; 420, 421 and 422 with two further groups of ions of lower intensity at: 462, 463 and 464; 476, 477 and 478 and for each of these groups the most intense peak is underlined.

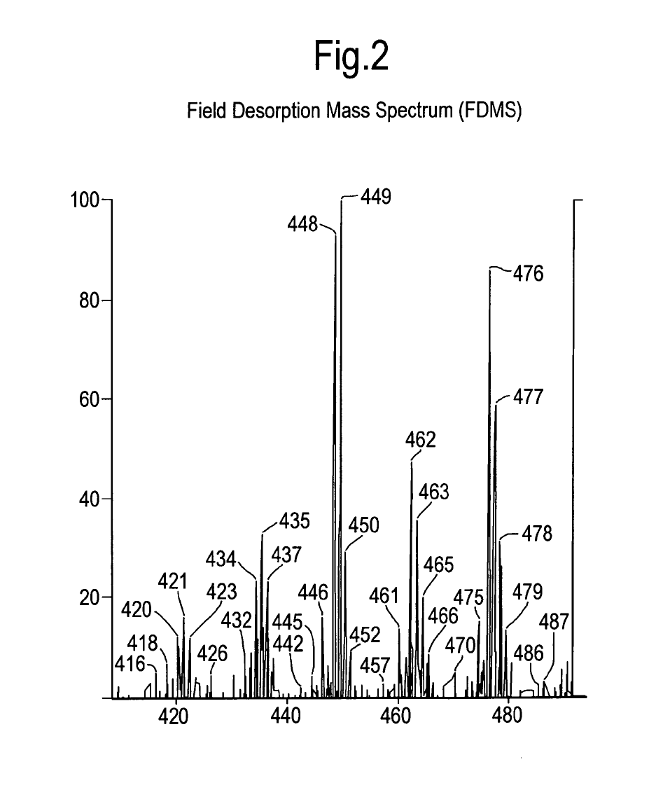

FIG. 2 shows the mass spectrum of the sample obtained using field desorption (FDMS). A similar pattern of peak envelopes is observed but with differences in relative intensities Compared to the FIMS. In this case the largest peaks were at 449 and 476. On the basis of analysis of the hydrocarbon sample prior to hydrogenation, which indicated the main component to be a botryococcene of formula C₃₄H₅₈ and molecular weight of 466, it is expected that the predominant molecule in the hydrogenated sample is C₃₄H₇₀ with a molecular weight of 478. A molecular ion (M⁺) of this mass was seen by FDMS, and to a much lesser extent in FIMS. However, with both of these ionisation techniques the main ion in this region appears to be at 476 mass units, corresponding to [M−2H]⁺(i.e. a molecular ion minus two protons). In FD the [M−H]⁺ion at 477 also appears to be larger than M+ at 478. The formation of [M−H]⁺and [M−2H]⁺ions by field-induced ion chemistry during FDMS and FIMS analysis of alkanes has been reported in Journal of Mass Spectrometry; Vol 31, 383-388 (1996); G. Klesper and F. W. Rollgen.

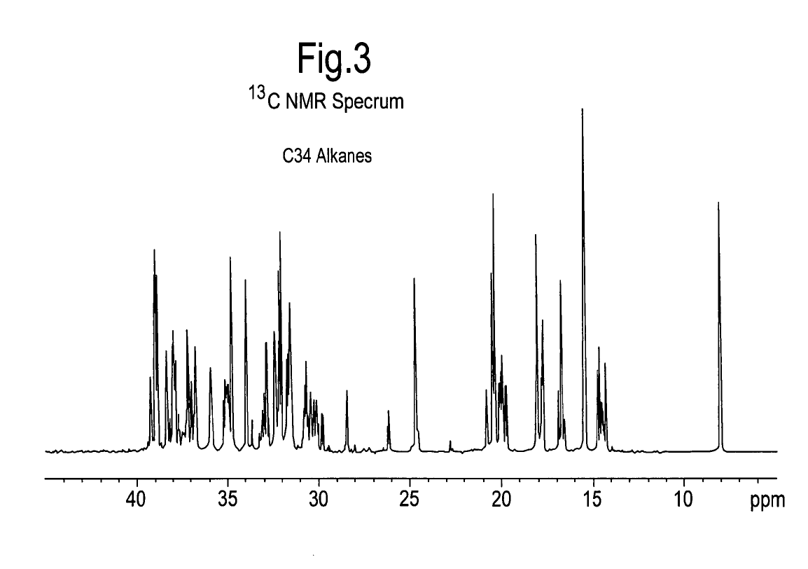

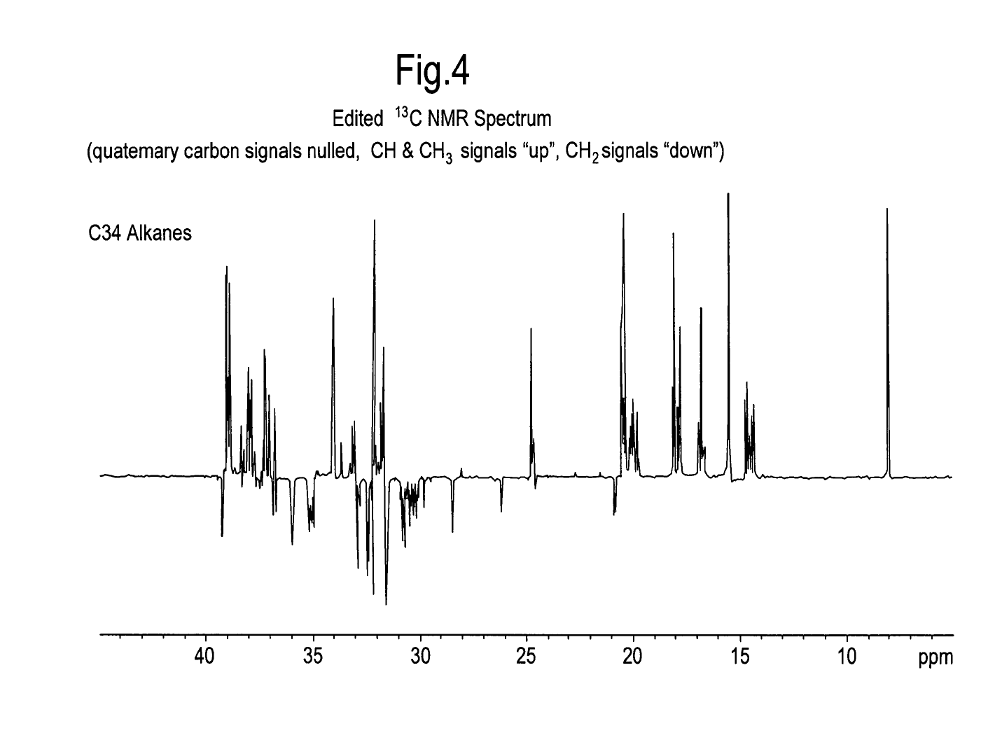

FIG. 3 shows the normal, proton-decoupled ¹³C NMR spectrum of the hydrogenated sample and FIG. 4 shows a similar spectrum obtained with spectral editing to differentiate the types of carbon atom. It is important to note that no signals are observed above 40 ppm, indicating that the sample contains no unsaturated carbons and that the hydrogenation reaction proceeded to completion (as also shown by ¹H NMR). The ¹³C NMR spectra are consistent with the view that the sample comprises predominantly a mixture of C₃₄, C₃₃ and C₃₂ botryococcanes. However, there are some peaks of low intensity which can not be accounted for by these botryococcanes and it is possible that these peaks are due to other saturated hydrocarbons that are present in small quantities. Such minor components could also account for the other signals observed by FIMS and FDMS (e.g. the signal groups around 421 and 435 mass units).

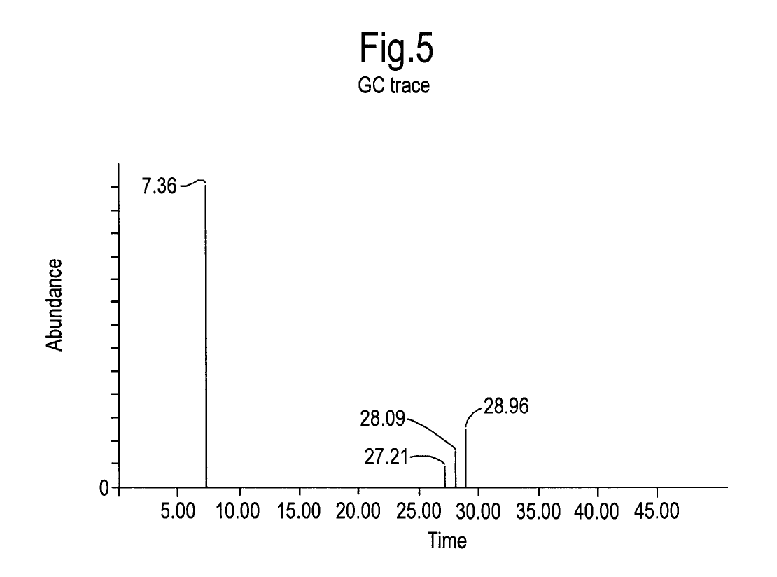

FIG. 5 shows a GC trace of GC-MS data of the hydrogenated sample containing 3 peaks between 27 and 29 minutes with an area ratio of 8%:26%:66%. The most likely assignment of these 3 GC peaks is to the C₃₂, C₃₃, and C₃₄ botryococcanes. These assignments are supported by the electron-impact MS data associated with each peak (not shown) in which the fragment ions can be rationalised in terms of the different molecular structures of the botryococcane homologues. Close inspection of the FIMS spectrum of the hydrocarbon prior to hydrogenation shows that, in addition to the botryococcene of molecular weight 466, there are also significant ions at 452 (corresponding to C₃₃H₅₆) and 438 (C₃₂H₅₄). These molecules produce, on hydrogenation the C₃₂ and C₃₃ botryococcanes.

### DETAILED DESCRIPTION OF THE INVENTION

The present invention relates to a novel base oil composition comprising at least one or more hydrogenated polymethylated triterpenes of the general formula CₙH₍₂ₙ₋₁₀₎.

Preferably, the hydrogenated polymethylated triterpenes comprise C₃₂, C₃₃ and C₃₄-botryococcanes, more preferably derived from living algae, more specifically from a Botryococcus braunii culture Race B.

The alga B. braunii is a small photosynthetic micro-organism that is widely distributed in fresh and brackish water, often occurring as a floating, green mat of cells.

The Botryococcus algae family are primitive colonial photosynthetic organisms, and may be regarded as a living fossil. For instance, oil shale deposits are populated with botryococcite fossils from which petroleum deposits arose.

B. braunii produces large amounts of hydrocarbons (up to 75% of the algal dry cell mass) from carbon dioxide, sunlight, water and inorganic mineral salts. B. braunii are usually divided into three races (A, B and L), differentiated by the main hydrocarbons produced, as described in Banerjee et al. (Critical Reviews in Biotechnology 22, 245-279, see below for a detailed discussion).

For decades, Botryococcus braunii has been suggested as a potential source of liquid transport fuels. In “Effect of media and culture conditions on the growth and hydrocarbon production by Botryococcus braunii”, Process Biochemistry 40 (2005) 3125-3131, C. Dayananda et al have proposed the use of the hydrocarbons derived from B. braunii as a refinery feedstock. The document describes that the hydrocarbons are removed from the algal cells either by solvent extraction, or after thermochemical liquefaction. Then the isolated hydrocarbons are subjected to catalytic cracking to produce gasoline. A further publication, GB-A-2423525, describes a process to yield biodiesel fuel from the biolipids derived from the biomass of race A of B. braunii algae.

Applicants have now found that the branched alkenes produced by B. braunii Races B and L (botryococcenes and lycopadiene, respectively) could be extracted and subjected to a hydrogenation without cracking. The resulting C₃₀ to C₄β iso-paraffins were found to be suitable for use as base oil components for lubricant compositions.

The microbiology, hydrocarbon production, cultivation and possible biofuel use of B. braunii have been reviewed in detail by Banerjee et al. (Banerjee A., Sharma R., Chisti Y. and Banerjee U. C. (2002)). Botryococcus braunii may be divided into three races (A, B and L), differentiated by the main hydrocarbons produced.

Race A produces predominantly hydrocarbons comprising C₂₃ to C₃₃ odd-numbered linear n-alkadienes and trienes (see formula I and II), at a maximum reported level of 60% wt. on the dry cell mass.

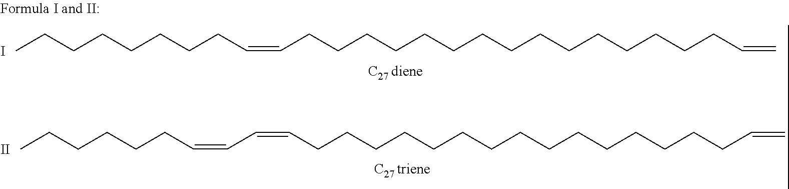

Race B produces hydrocarbons comprising C₃₀ to C₃₇ and predominantly C₃₂-C₃₄ polymethylated triterpenes, (also referred to as ‘botryococcenes’) of the general formula and C₃₁-C₃₄ methylated squalenes, at a maximum CₙH₍₂ₙ₋₁₀₎, reported level of from 25-85% wt. on dry cell mass (see formula III and IV, respectively).

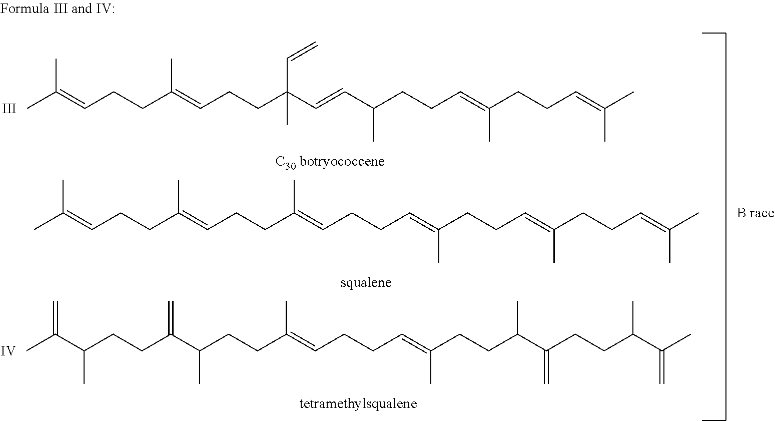

Race L comprises predominantly an acyclic C₄₀H₇₈ tetraterpene (referred to as ‘lycopadiene’) at a maximum reported level of 2-8% wt. dry cell mass (see formula V).

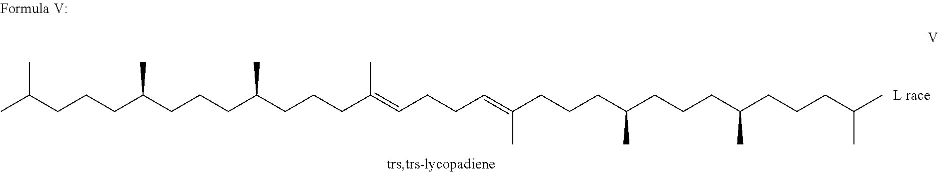

Applicants found that the isolated branched alkenes produced by B. braunii Races B and L, i.e. botryococcenes and lycopadiene, respectively, were hydrogenated under conditions that avoid significant amounts of cracking, then this resulted in C₃₀ to C₄₀ iso-paraffin mixtures. These branched alkanes were found highly suitable for use as lubricant base stocks. Accordingly, the present invention also relates to a lubricant composition comprising a base oil composition according to the invention, and at least one additive, and to the use of a base oil derived from B. braunii in a lubricant for the increase of oxidation stability.

Accordingly, the invention also relates to a process for the preparation of a base oil, comprising (a) extracting hydrocarbons from the alga B. braunii Race B, and (b) hydrogenating the extracted hydrocarbons, and (c) isolating the hydrogenated and extracted hydrocarbons to obtain the base oil composition according to the subject invention. Preferably, the process also includes a further step of cultivating the alga B. braunii Race B.

Step (a) can be performed in any manner that is suitable for isolating the hydrocarbons from the algal cells, as the methods disclosed on page 270 ff of B. braunii: A Renewable source of Hydrocarbons and Other Chemicals (Banerjee A., Sharma R., Chisti Y. and Banerjee U. C. (2002)). Step (a) may thus comprise the steps of (a1) rupturing the algal cells; (a2) separating the hydrocarbons from ruptured cell material. Alternatively, step (a) may be applied in such manner that the hydrocarbons are extracted from the cells by a suitable medium without rupturing the cell membrane, e.g. by solvent extraction.

Step (b) may be performed in any manner suitable to hydrogenate the hydrocarbons isolated in step (a). Preferably, step (b) is performed in such away that any cracking or reforming reactions are minimized. More preferably, step (b) is performed such that less than 25% wt. of the product boiling above 300° C. is cracked away, yet more preferably less than 20% wt. of the product boiling above 300° C. is cracked away, and most preferably less than 15% wt. of the product boiling above 300° C. is cracked away. The term “cracked away” means that the products having such boiling ranges are cracked to lower boiling products and to gas. This may suitably done in solution in an inert solvent, such as n-hexane or similar solvents.

Suitably, step (b) is performed under mild conditions in the presence of a hydrogenation catalyst comprising a hydrogenation component, and hydrogen. It has appeared that especially a metal selected from group VIII (of the periodic table of elements) catalyst on a wide-pore alumina is able to hydrogenate such compounds in such a way that all unsaturations are removed.

The hydrogenation catalyst preferably comprises a metallic active portion in which the metal is a non-noble Group VIII metal and a support, characterised in that the support does not catalyse an acid catalysed reaction and wherein over 90% of the pores within the support are sized between 10 nm to 40 nm.

The support preferably has a sharp pore size distribution. Over 90% of the pores within the support are sized between 10 nm to 40 nm. Preferably over 70% of the pores are sized between 12 nm to 35 nm.

Typically the median pore diameter is around 12 nm, preferably greater than 12 nm. More preferably the median pore diameter is around 15 nm, even more preferably over 17 nm, around 19 nm. Preferably less than 25%, more preferably less than 11% of the pore volume is provided by pores with a diameter greater than 35 nm. Even more preferably less than 8% of the pore volume is provided by pores with a diameter greater than 35 nm. In some embodiments less than 6% of the pore volume is provided by pores with a diameter greater than 35 nm.

The pore volume is determined using the Standard Test Method for Determining Pore Volume Distribution of Catalysts by Mercury Intrusion Porosimetry, ASTM D 4284-88.

Preferably the support comprises wide pore alumina, more preferably the wide pore alumina disclosed in U.S. Pat. No. 4,248,852 and which is incorporated herein by reference in its entirety. Alternatively wide pore alumina, as disclosed in U.S. Pat. No. 4,562,059, may also be used. The preparation of the support may be as described in U.S. Pat. No. 4,422,960. U.S. Pat. Nos. 4,562,059 and 4,422,960 are incorporated herein by reference in their entirety. Preferably the active portion comprises a group VIII metal, such as nickel, cobalt or molybdenum, or combinations thereof. Preferably the catalyst comprises less than 20% wt. of the metal, and preferably more than 5% wt. of the metal, nickel. Preferably the active component comprises a dopant to suppress hydrogenolysis of paraffins to methane. Copper is one example of a suitable dopant. The active portion is preferably substantially pure nickel with the dopant but can be, for example, nickel/molybdenum, nickel with palladium or platinum, and can be a nickel sulphide, a nickel molybdenum sulphide, or a nickel tungsten sulphide.

Alternatively the active portion may comprise noble metals such as palladium or platinum; cobalt, cobalt/molybdenum, cobalt/molybdenum sulphide.

Preferably the catalyst is adapted to hydrogenate olefins. More preferably the catalyst is adapted to hydrogenate oxygen-containing compounds and olefins.

During manufacture, preferably the active portion is impregnated onto the support. The method for manufacturing the hydrogenation catalyst as described above preferably comprises: admixing a solution of a metal salt with a support; drying and calcining the mixture. More preferably the metal is impregnated into the support.

Typically the method produces a catalyst with metal oxide particles on the support and the metal oxide is reduced in situ before the catalyst is used. Preferably the metal salt is mixed in a basic solution.

The invention will be further illustrated by the following, non-limiting examples:

Race B and Race L B. braunii strains were cultivated in a standard batch cultivation. Then the algal hydrocarbons were obtained by a standard solvent extraction using n-hexane as described by Frenz J., Largeau C., Casadevall E., Kollerup F. and Daugulis A. J. Hydrocarbon recovery and biocompatibility of solvents for extraction from cultures of Botryococcus braunii. Biotechnology and Bioengineering 34, 755-762 (2004).

### Example 1

### B. braunii Race B Hydrocarbon Hydrogenation

A 4 ml sample of B. braunii Race B hydrocarbons was subjected to hydrogenation.

200 mg of a Ni−Al₂O₃ hydrogenation catalyst comprising 18 wt. % of nickel on a theta-alumina carrier having a surface area of 110 m²/g were pre-activated by subjecting it to a hydrogen atmosphere at 10 bar of H₂ partial pressure for 10 hours at 190° C. Then 500 mg of a sample of B. braunii Race B hydrocarbons were dissolved in 3 ml n-hexane, and added to the catalyst at a hydrogen partial pressure of 30 bar, and the mixture was stirred for 10 hours at 190° C. ¹H- and ¹³C-NMR spectroscopy of the product indicated that only trace amounts of unsaturation remained.

The catalyst was filtered off, the solvent removed and the product was isolated as liquid at ambient conditions.

### Analysis of Saturated Algal Hydrocarbons

The obtained sample was analysed to determine its composition. The analysis was performed using standard GC-FIMS and ¹³C-NMR analyses, as set out below.

A range of analytical data collectively indicate that the sample is predominantly a mixture of C₃₄, C₃₂ and C₃₃ botryococcanes but with also a small proportion of other saturated hydrocarbon molecules. Gas chromatography (GC) and field-ionisation mass spectrometry (FIMS) were used to confirm the presence of particular hydrocarbons in extracts of Race B and Race L of B. braunii.

Analysis by mass spectrometry was carried out using a Finnigan MAT90 Mass Spectrometer to perform Field Desorption and Field Ionisation Mass Spectrometry. For ¹³C NMR the sample was dissolved in approximately 0.5 ml of deuterochloroform and analysed on a Bruker Avance 400 spectrometer. A spectrum was also obtained with a DEPT-135 pulse sequence, by which quaternary carbon signals are eliminated and —CH— and —CH₃ signals appear with the opposite phase to —CH₂ signals. This demonstrated a predominance of C₃₂-C₃₄ botryococcanes according to formula VI (i.e. saturated, uncracked algal hydrocarbons):

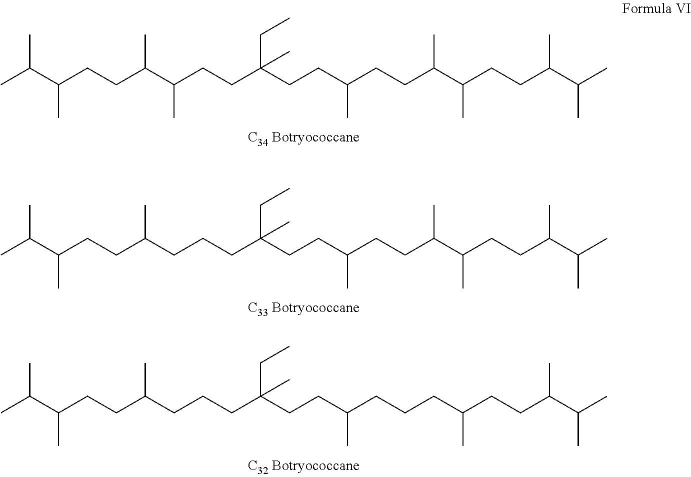

Properties of Saturated B. braunii Race B Algal Hydrocarbons as Base Oils in Lubricant Compositions
The properties of saturated B. braunii Race B algal hydrocarbons as a base oil component for a lubricant formulation were evaluated as follows, in comparison to reference mineral base oil samples:

Viscosity vs. temperature (−20° C. to 100° C.);

ISO viscosity grade;

Viscosity index;

Pour point; and

Oxidative stability.

### Comparative Example 1

### Viscometric Properties

The dynamic viscosity and change in viscosity with temperature (−20° C. to 100° C.) of the sample were determined using a temperature-controlled cone and plate rheometer (TA Instruments, TA1000 stress-controlled rheometer). Molecular modelling (Advanced Chemistry Inc. ACD/ChemSketch) of C₃₂₋₃₄ botryococcanes yielded a density of 0.81 g/ml; this enabled kinematic viscosity at 40° C. and 100° C., and viscosity index (VI), to be calculated from the dynamic viscosity data. The pour point was estimated from when the sample began to form an elastic structure as this indicates the onset of solidification at low temperatures.

This method of estimating pour point was validated using standard mineral oils of known pour points which had measured using the standard procedure (e.g. ASTM D 97, ISO 3016).

The viscometric properties for the saturated B. braunii Race B algal hydrocarbons are shown in Table 1.

TABLE 1
Viscometric properties for the saturated
B. braunii Race B algal hydrocarbons

| PROPERTY | VALUE |
|---|---|
| Kinematic viscosity (mm²/s) at: | |
| 0° C. | 1589 |
| 40° C. | 74 |
| 100° C. | 9 |
| ISO Viscosity Grade (ISO 3448) | ISO VG 68 |
| Viscosity index (ISO 2909) | 90 |
| Estimated pour point (° C.) | below −20 |

The data shown in Table 1 indicate that the saturated B. braunii Race B algal hydrocarbons had kinematic viscosities and a viscosity index (change in viscosity with temperature) comparable to paraffinic mineral base oils; whilst cold temperature flow (pour point) was significantly lower (i.e. better). These features indicated that the sample was suitable for lubricant base oil use.

### Comparative Example 2

### Oxidative Stability

Lubricant compositions were prepared from several base oils. The oxidative stability of saturated B. braunii Race B hydrocarbons, when supplemented with two commonly used aminic and phenolic antioxidant additives, was compared with representative API (American Petroleum Institute) Group II, III and IV base oils of a similar viscosity (around 8 mm²/s at 100° C.). Oxidative stability of the lubricant compositions was measured by pressure differential scanning calorimetry (PDSC) using a Mettler/Toledo HP DSC 827 instrument and the following test conditions: isothermal at 160° C., 200 psig, zero flow O₂ atmosphere, 2.00±0.05 mg sample and 40 μl Al pans. A longer oxidation induction period in this test indicates a greater oxidative stability of the test sample.

The response (oxidative stability) of the saturated Race B hydrocarbons to the aminic antioxidant Irganox L57® (ex. Ciba) was found to be significantly better than the reference base oils. The reference base oils were an API Gp II STAR 8 base oil (commercially available from Motiva), a catalytically dewaxed Fischer-Tropsch GP III base oil, and an API group IV Durasyn 168 base oil (commercially available from Innovene).

Table 2 depicts the results.

TABLE 2
Oxidative stability of the saturated B. braunii Race
B algal hydrocarbons and representative base oils when
inhibited with phenolic and aminic antioxidants

| | | | |
|---|---|---|---|
| | | | OXIDATION |
| | | ANTIOXIDANT | INDUCTION |
| API | | (0.5% wt. | PERIOD |
| GROUP | BASE OIL | treat rate) | (minutes) |

Phenolic antioxidant

| | | | |
|---|---|---|---|
| II | STAR 8 (commercially | HiTEC 4702 ® | 23 |
| | available from Motiva) | (ex. Afton) | |
| III | XHVI 8 (commercially | HiTEC 4702 ® | 22 |
| | available from. Shell) | (ex. Afton) | |
| IV | Durasyn 168 (XHVI 8 | HiTEC 4702 ® | 27 |
| | (commercially | (ex. Afton) | |
| | available from | | |
| | Innovene) | | |
| — | Saturated B. braunii | HiTEC 4702 ® | 17 |
| | Race B hydrocarbons | (ex. Afton) | |

Aminic antioxidant

| | | | |
|---|---|---|---|
| II | STAR 8 | Irganox L57 ® | 46 |
| | | (ex. Ciba) | |
| III | XHVI 8 | Irganox L57 ® | 29 |
| | | (ex. Ciba) | |
| IV | Durasyn 168 | Irganox L57 ® | 44 |
| | | (ex. Ciba) | |
| — | Saturated B. braunii | Irganox L57 ® | 68 |
| | Race B hydrocarbons | (ex. Ciba) | |

### Comparative Example 3

### B. braunii Race L Hydrocarbons

Samples of the Race L alkenes were hydrogenated using the procedure of Example 1. The hydrogenated sample was a solid at room temperature, and therefore unsuitable for use as a lubricant base oil.

1. A base oil composition comprising at least one or more hydrogenated polymethylated triterpenes of the general formula CₙH₍₂ₙ₊₂₎.

2. A base oil composition according to claim 1, wherein the hydrogenated polymethylated triterpenes comprise C₃₂, C₃₃ and C₃₄-botryococcanes.

3. A base oil composition according to claim 1 or claim 2, wherein the botryococcanes are derived from living algae.

4. A base oil composition according to claim 3, wherein the botryococcanes are derived from a Botryococcus braunii culture Race B.

5. A composition according to any one of claims 1 to 4, having a viscosity in the range of from 4 to 12 cSt (mm²/s)

6. A lubricant composition comprising a base oil composition according to claims 1 to 5, and at least one additive.

7. Use of a base paraffinic base oil derived from Botryococcus braunii according to claims 1 to 5 in a lubricant for the increase of oxidation stability.

8. A process for the preparation of a base oil, comprising
(a) extracting hydrocarbons from the living alga Botryococcus braunii Race B, and
(b) hydrogenating the extracted hydrocarbons, and
(c) isolating the hydrogenated and extracted hydrocarbons to obtain the base oil composition.

9. A process according to claim 8, wherein step (b) is performed such that less than 25% wt. of the product boiling above 300° C. is cracked away.

10. A process according to claim 8 or 9, comprising a further step of cultivating the alga Botryococcus braunii Race B.
